(19) United States
(12) Patent Application Publication
(10) Pub. No.: US 2006/0263389 A1
(43) Pub. Date: Nov. 23, 2006

(54) Methods and compositions to enhance immune responses via recall antigens

(76) Inventors: Sue Carol Stacy, San Antonio, TX (US); Ellen Beth Kraig, San Antonio, TX (US); Peter Dube, San Antonio, TX (US)

Correspondence Address: MYERS BIGEL SIBLEY & SAJOVEC, PO BOX 37428, RALEIGH, NC, 27627, US

(21) Appl. No.: 11/326,869

(22) Filed: Jan. 7, 2006

Related U.S. Application Data
(63) Continuation-in-part of application No. PCT/US04/22734, filed on Jul. 7, 2004.
(60) Provisional application No. 60/485,615, filed on Jul. 9, 2003.

(51) Int. Cl. A61K 39/02; A61K 39/08; A61K 48/00

(52) U.S. Cl. 424/234.1; 424/239.1; 514/44

(57) ABSTRACT

The present invention provides a composition comprising a chimeric polypeptide comprising a recall antigen that reactivates memory T cells in a subject and a new antigen that activates naïve B cells in a subject and a composition comprising a nucleic acid encoding a chimeric polypeptide comprising a recall antigen that reactivates memory T cells in a subject and a new antigen that activates naïve B cells in a subject. Further provided are methods of modulating an immune response, as well as treating and/or preventing disease in subjects in whom the ability to mount an immune response to a new antigen is impaired, by administering the compositions of this invention.

### STATEMENT OF PRIORITY

The present application is a continuation-in-part application and claims priority to International Application Serial No. PCT/US2004/022734, filed Jul. 7, 2004, and published in English as PCT Publication No. WO 2005/005465 A2 on Jan. 20, 2005, which claims the benefit of U.S. provisional application Ser. No. 60/485,615, filed Jul. 8, 2003, the entire contents of each of which are incorporated by reference herein.

### STATEMENT OF GOVERNMENT SUPPORT

Research directed to this invention is supported in part by NIH Grant No. 1 RO3 AG022675-01, awarded by the National Institutes on Aging/National Institutes of Health. The United States Government has certain rights in this invention.

### BACKGROUND OF THE INVENTION

It has been well documented that aging negatively impacts the immune system; this results in decreased effectiveness of vaccination and increased susceptibility to infectious diseases and cancer (reviewed in 1-4,10-12). Much of the loss in immune competence with age is due to involution of the thymus. Since most T cells undergo development and T cell receptor (TCR) gene rearrangement in the thymus, the loss of its functional capacity with age results in a significant decline in the production of naïve T cells (13). In fact, recent studies in humans have documented a ≧10-fold decrease in thymic emigration of mature T cells over the lifetime of an individual (14). Thus, the repertoire of naïve T cells available to respond to antigens not previously seen is reduced and T cell responses become compromised. Moreover, those few naïve T cells that are produced in the elderly individual appear to be less responsive. This has been studied in transgenic mice in which almost all T cells express the same TCR directed against one antigenic epitope. If the TCR transgenic mouse is never exposed to the relevant antigen, all the T cells remain phenotypically naïve (15). Studies using such mice have shown that naïve T cells in older animals are indeed compromised with regard to proliferation responses, IL-2 production, and the ability to differentiate into effector cells (15). It is significant that these defects can be partially overcome by the administration of the cytokine, IL-2 (15), especially in view of the shifts in cytokine profiles seen in the elderly. In general, an age-associated shift towards a T-helper2 (Th2) phenotype is seen in both humans and rodents (16-18); that is, IL-2 and IL-2 receptor levels decline while IL-4 levels increase (17). Thus, the trend in general cytokine responses does not help to compensate for deficiencies in naïve T cells in the elderly. This decreased production of naïve T cells with age coupled with a lifetime exposure to antigen results in an increased predominance of memory T cells in the older individual. Thus, many of the age-associated alterations that have been reported in T cell function (19-22) may be due, at least in part, to this shift towards memory T cells. While T cell responses of total populations of T cells and of mixtures of naïve T cells have been often studied, less is known about specific alterations in the memory T cell compartment in older individuals (23-25).

Diminished or altered T cell “help” in older individuals plays a major contributory role to the alterations observed in B cell responses (1,26). Although the overall number of B cells changes little with age, declines in antibody responses to antigenic challenge have been widely reported (10,27-29). Associated with the overall decrease in antibody production is a shift in the expressed immunoglobulin variable region repertoire (27) and diminished somatic mutation (30). Both of these most likely contribute to the age-associated decrease in antibody affinity in older immunized mice (30,31). Adoptive transfer experiments have shown that these B cell events are controlled, in part, by T cells. In addition, the diversity of the overall B cell repertoire is reportedly diminished in older individuals (2). For example, recovery of the lymphocytic compartment after cytotoxic drug treatment resulted in a less diverse B cell repertoire in older animals. It is suggested that this may result from impairment of B cell development in the bone marrow (2,32,33). Moreover, studies using BrdU labeling suggest a decline in the output of newly processed B cells into the periphery (34). Nevertheless, the number of peripheral lymphocytes remains relatively stable, possibly due to an increase in their life span with age (2).

Because naive B cell responses in old animals are diminished in terms of titer and affinity, it is not surprising that memory immunity which develops from these “aging” naive responses would also be less effective (27,35,36). It has been shown, however, that memory B and T cells produced at a young age can persist for years, in some cases for a lifetime (37) and data from various animal studies suggest that memory cells developing in an individual's youth survive aging and produce a relatively vigorous response if re-activated in old age (6-9,38).

Numerous strategies have been employed to improve the effectiveness of vaccines particularly for use in the very old or very young. For example, polysaccharide (T cell independent antigens) can be used for effective vaccines in infants only if they are conjugated to a proteinaceous T cell dependent antigen (TD). This approach relies on T cell “help” from the TD antigen to boost the B cell response to the polysaccharide (39). Sanchez et al. reported that B cell responses to TI antigens in old mice were boosted by conjugating the TI antigen to a TD antigen, but only if the mice had first been primed with the conjugate at a young age (6).

Various other strategies to enhance immune responses to vaccination in the elderly have been investigated. In attempts to enhance the immune response the antigenic agent in influenza vaccines has been doubled (41). In another study, live attenuated influenza virus was combined with a trivalent subunit inactivated vaccine (42). Alternatively, using a mucosal route of administration has been suggested in order to enhance responses in the respiratory mucosa (43,44). Others have suggested administering reagents that stimulate antigen presenting cell function (45). Animal experiments have utilized the adjuvant effect of CpG motifs to enhance immunization responses in the elderly mice (46). However, these and other approaches to enhancing immune responses to vaccination in elderly humans have been very limited in their effectiveness (41,42).

The present invention overcomes previous problems in the art by providing methods and compositions that improve and enhance vaccine efficacy in subjects in whom the ability to mount an immune response to a naïve antigen is impaired (e.g., the elderly).

### SUMMARY OF THE INVENTION

The present invention provides a composition comprising a chimeric polypeptide comprising a recall antigen that reactivates memory T cells in a subject and a new antigen that activates naïve B cells in a subject.

Further provided is a composition comprising a nucleic acid encoding a chimeric polypeptide comprising a recall antigen that reactivates memory T cells in a subject and a new antigen that activates naïve B cells in a subject.

An additional aspect of the present invention provides a composition comprising a chimeric polypeptide comprising a recall antigen that reactivates memory T cells in a subject and a new antigen that activates naïve B cells in a subject, wherein the recall antigen is tetanus toxoid fragment C (TTFC) and the new antigen is Yersinia pestis LcrV antigen (V antigen).

A further embodiment of the present invention provides a composition comprising a nucleic acid encoding a chimeric polypeptide comprising a recall antigen that reactivates memory T cells in a subject and a new antigen that activates naïve B cells in a subject, wherein the recall antigen is tetanus toxoid fragment C (TTFC) and the new antigen is Yersinia pestis LcrV antigen.

The present invention additionally provides various methods employing the compositions of this invention, including, for example, a method of eliciting an immune response to a new antigen in a subject in whom the ability to elicit an immune response to a new antigen is impaired, comprising administering to the subject an effective amount of the compositions of this invention.

Also provided is a method of treating a disorder and/or preventing a disease in a subject in whom the ability to elicit an immune response to a new antigen is impaired, comprising administering to the subject an effective amount of the compositions of this invention.

In further embodiments, the present invention provides a method of eliciting an immune response to a new antigen in a subject in whom the ability to elicit an immune response to a new antigen is impaired, comprising: a) identifying a recall antigen that reactivates memory T cells in the subject; b) optionally identifying a new antigen against which the subject has little or no detectable memory immune response; and c) administering to the subject an effective amount of a composition comprising a chimeric polypeptide comprising the recall antigen of step (a) and a new antigen.

Further provided herein is a method of eliciting an immune response to a new antigen in a subject in whom the ability to elicit an immune response to a new antigen is impaired, comprising: a) identifying a recall antigen that reactivates memory T cells in the subject; b) optionally identifying a new antigen against which the subject has little or no detectable memory immune response; and c) administering to the subject an effective amount of a composition comprising a nucleotide sequence encoding a chimeric polypeptide comprising the recall antigen of step (a) and a new antigen.

The present invention additionally provides a method of treating a disorder in a subject in need of such treatment and/or preventing a disorder in a subject in whom the ability to elicit an immune response to a new antigen is impaired, comprising: a) identifying a recall antigen that reactivates memory T cells in the subject; b) optionally identifying a new antigen against which the subject has little or no detectable memory immune response and to which an immune response can be produced that is effective in treating the disorder; and c) administering to the subject an effective amount of a composition comprising a chimeric polypeptide comprising the recall antigen of step (a) and a new antigen.

Furthermore, the present invention provides a method of treating a disorder in a subject in need of such treatment and/or preventing a disease in a subject in whom the ability to elicit an immune response to a new antigen is impaired, comprising: a) identifying a recall antigen that reactivates memory T cells in the subject; b) optionally identifying a new antigen against which the subject has little or no detectable memory immune response and to which an immune response can be produced that is effective in treating the disorder; and c) administering to the subject an effective amount of a composition comprising a nucleotide sequence encoding a chimeric polypeptide comprising the recall antigen of step (a) and a new antigen.

### BRIEF DESCRIPTION OF THE DRAWINGS

FIGS. 1A-B show the effects of aging on the B cell response to T-AChR (antigen from Torpedo californica). FIG. 1A: Mice in three age groups were given multiple immunizations with T-AChR/CFA, one month apart. The sera from primary, secondary, and tertiary exposures were subjected to serial dilutions and analyzed by ELISA for reactivity to T-AChR. The titers were defined as the dilution when the half maximum O.D. was obtained. In order to assess the recall memory response, young mice were given multiple immunizations and then aged without further exposure to T-AChR until they were 18 months old. After a fourth immunization, serum samples were obtained and analyzed as described herein. FIG. 1B: Antibody isotype levels were measured by ELISA using serum samples taken four weeks post primary T-AChR immunization from 4 young and 8 old mice and diluted 1/125. Statistically significant differences (p=0.05) are indicated by the asterisks.

FIGS. 2A-B show the effects of aging on heterogeneity of the anti-T-AChR response. FIG. 2A: Sera were prepared from mice immunized with T-AChR at either two months (young) or 20 months (old). The heterogeneity of the antibody responses in young and old mice were assessed by isoelectric focusing (IEF) coupled to immunoblotting on paper saturated with T-AChR. In this way, very heterogeneous responses (like those seen in the young animals) can be distinguished from more restricted B cell responses (seen in some, but not all, of the older animals). FIG. 2B: The clonotypic profile of the anti-T-AChR antibody responses in sera extracted from 2-4 month old mice after primary and tertiary immunization is compared with the profile after a recall immunization at 18 months of age. The IEF/immunoblotting protocol is described herein.

FIGS. 3A-B show the production of a recombinant T-AChR α fragment. A PCR product containing codons 1-210 of the T-AChRα chain was cloned into pRSET-A and expressed in E. coli. FIG. 3A: The bacterial extract was subjected to chromatography on NTA-agarose and the fusion protein was analyzed on a Western blot with an antibody to the His-tag. FIG. 3B: The His-AChR α was subjected to denaturation and renaturation. This was compared to unrenatured recombinant AChRα and to a negative control (recombinant CREB-2) using an ELISA with sera from T-AChR immunized mice.

FIG. 4 illustrates the T-AChRα-TTFC expression plasmid. The 1374-bp TTFC encoding nucleic acid is generated by PCR using primers with restriction sites incorporated into their sequence (forward: 5′-CCCAGGTACCTCAACACCAATTCCATTTT3′ (SEQ ID NO:1) and reverse 5′-GTAGAATTCTGTCCATCCTTCATCTGTA3′ (SEQ ID NO:2)). The PCR-generated TTFC nucleic acid is cut with KpnI and EcoRI and inserted in frame into the pEK682 also digested with KpnI and EcoRI. The plasmid pEK682 contains a 639 bp gene fragment encoding for aa1-210 from the extracellular portion of the T-AChRα chain and flanked by a KpnI site. The plasmid is transformed into BL21 (lysS) and expression induced by addition of IPTG.

### DETAILED DESCRIPTION OF THE INVENTION

As used herein, “a” or “an” or “the” can mean one or more than one. For example, “a” cell can mean one cell or a plurality of cells.

Also as used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

As used herein, “modulate,” “modulates” or “modulation” refers to enhancement (e.g., an increase) or inhibition (e.g., diminished, reduced or suppressed) of the specified activity. The term “enhancement,” “enhance,” “enhances,” or “enhancing” refers to an increase in the specified parameter (e.g., at least about a 1.1-fold, 1.25-fold, 1.5-fold, 2-fold, 3-fold, 4-fold, 5-fold, 6-fold, 8-fold, 10-fold, twelve-fold, or even fifteen-fold or more increase). The term “inhibit,” “diminish,” “reduce” or “suppress” refers to a decrease or reduction in the specified activity of at least about 5%, 10%, 25%, 35%, 40%, 50%, 60%, 75%, 80%, 90%, 95%, 97%, 98%, 99% or 100%. In particular embodiments, the inhibition or reduction results in little or essentially no detectible activity (at most, an insignificant amount, e.g., less than about 10% or about 5%).

The present invention is based on the unexpected discovery that memory T cells generated at a young age survive aging, allowing these cells to be exploited to generate effective antibody (B cell) responses to “new” antigens first seen in animals, such as older animals, that have become impaired in their ability to mount an effective immune response to a new antigen.

Thus, in one embodiment, the present invention provides a chimeric polypeptide comprising, consisting essentially of and/or consisting of, a “recall” antigen (one that re-activates memory T cells in a subject) fused to a “new” antigen (one that invokes or activates a response by “old” naïve T and B cells). The recall antigen stimulates “youthful” memory T cells and these T cells provide the robust “recall help” to naive B cells responding to a newly introduced antigen.

As used herein, a “recall antigen” is an antigen to which a subject has had prior immune exposure and against which memory T cells exist in the subject that recognize the recall antigen. Also as used herein, a “new antigen” or “naïve antigen” is an antigen to which a subject has had little or no prior exposure that resulted in an immune response that can be detected by methods standard in the art for detecting an immune response, such as enzyme immunoassay (EIA) and/or ELISA. As used herein, “little or no prior exposure” can mean an undetectable or reduced amount or level of immune response to an antigen demonstrated by a subject as compared to the amount or level of immune response typically seen in a normal subject (e.g., a subject without any identified or suspected impairment in that ability to elicit an effective and/or measurable immune response) who has previously generated a response to that antigen. A new antigen of this invention can also be any antigen identified to be appropriate for administration to a subject of this invention to achieve a particular prophylactic and/or therapeutic effect.

A subject of this invention is a subject that is identified to be or suspected to be impaired in the ability to elicit an effective and/or measurable immune response to a new antigen. The subject, which can be a mammal such as a human, can mount an effective and/or measurable memory response to a recall antigen (as compared to a normal subject) but has a diminished ability to mount an effective and/or measurable immune response to a new or naïve antigen in comparison to an immune response to a new or naïve antigen identified and/or measured in a normal subject. Thus, in certain embodiments, the subject of this invention is most likely to be an old subject and is typically not a subject who is unable to mount an effective memory response (e.g., an immunocompromised subject such as an HIV-infected subject who can no longer mount an effective memory response). As noted above, the subject can be a human, or the subject of this invention can be any animal with an immune system that produces memory T cells activated by recall antigens and that assist in the production of antibodies to new antigens.

A subject of this invention may also be a subject that is in need of suppression of a particular immune response. For example, the subject may be in need of treatment and/or preventino of an autoimmune disease and/or allergic response.

As used herein, “elicits an immune response” or “eliciting an immune response” includes the development, in a subject, of a humoral and/or a cellular immune response to a protein and/or polypeptide of this invention (e.g., an immunogen, an antigen, an immunogenic peptide, and/or one or more epitopes). This can also mean that the immune cells are reactive to compositions of the present invention and an immune response is generated in response thereto, which is modulated, e.g., enhanced or suppressed. In such a case where the modulation is suppressive in nature it can be used, for example, to treat an autoimmune and/or allergic response.

The identification of a recall antigen and/or a new antigen as those terms are used relative to a given subject of this invention is carried out by methods routine in the art for detecting an established immune response. Such methods can include, but are not limited to, antibody detection assays such as, for example, EIA (enzyme immunoassay), ELISA (enzyme linked immunosorbent assay), agglutination reactions, precipitation/flocculation reactions, immunoblots (Western blot; dot/slot blot); (RIA) radioimmunoassay, immunodiffusion assays, histochemical assays, immunofluorescence assays (FACS), chemiluminescence assays, library screens, expression arrays, etc. Assays for the detection of T cell responses include, but are not limited to, delayed-type hypersensitivity responses, in vitro T cell proliferation responses (e.g., measured by incorporation of radioactive nucleotides), library screens, expression arrays, T cell cytokine responses (e.g., measured by ELISA or other related immuno-assays or RT-PCR for specific cytokine mRNA), as well as any other assay known for measuring a B cell and/or T cell immune response in a subject.

Also as used herein, the term “chimeric polypeptide” includes a polypeptide comprising a first amino acid sequence (the recall antigen) that can be a peptide, a fragment of a protein or a whole protein that is linked or joined to a second amino acid sequence (the new antigen) that can be a peptide, a fragment of a protein or a whole protein and wherein the first and second amino acid sequences are not linked or joined in the same way in nature.

The recall antigen and new antigen can be immediately adjacent to one another on the chimeric polypeptide and/or separated by a spacer sequence of amino acids. The spacer sequence can be 1, 2, 3, 4, 5, 6, 7, 8, 9, 10, 11, 12, 13, 14, 15, 16, 17, 18, 19, 20, 21, 22, 23, 24, 25, 50, 60, 70, 80, 90, 100, 125, 150, 175, 200, or greater than 200 amino acids in length and can be any amino acids.

In one embodiment of the present invention, the spacer sequence separating a recall antigen and a new antigen can comprise, consist essentially of, and/or consist of, an enterokinase site, a sequence specific motif recognized by the enterokinase protease (DDDDR; SEQ ID NO:5). A further embodiment of the present invention provides a spacer sequence comprising, consisting essentially of, and/or consisting of, a peptide that comprises, consists essentially of, and/or consists of, the amino acid sequence of SEQ ID NO:6.

Additionally provided is a spacer sequence comprising, consisting essentially of, and/or consisting of, a nucleotide sequence comprising, consisting essentially of, and/or consisting of, a nucleotide sequence encoding the amino acid sequence of SEQ ID NO: 5 or SEQ ID NO:6.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP § 2111.03.

The recall antigen and the new antigen can be present in the chimeric polypeptide in a ratio of 100:1, 90:1, 80:1, 70:1, 60;1, 50:1, 40:1, 30:1. 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90 and/or 1:100 or more. Furthermore, a chimeric polypeptide of this invention can comprise more than one recall antigen and/or more than one new antigen. The chimeric polypeptide can comprise, consist essentially of, and/or consist of, multiple recall antigens that can be all the same, all different, or a combination thereof. The chimeric polypeptide can also comprise multiple new antigens that can be all the same, all different or a combination thereof.

Also as used herein, the terms peptide and polypeptide are used to describe a chain of amino acids, which correspond to those encoded by a nucleic acid. A peptide usually describes a chain of amino acids of from two to about 30 amino acids and polypeptide usually describes a chain of amino acids having more than about 30 amino acids. The term polypeptide can refer to a linear chain of amino acids or it can refer to a chain of amino acids, which have been processed and folded into a functional protein. It is understood, however, that 30 is an arbitrary number with regard to distinguishing peptides and polypeptides and the terms may be used interchangeably for a chain of amino acids around 30. The peptides and polypeptides of the present invention are obtained by isolation and purification of the peptides and polypeptides from cells where they are produced naturally or by expression of a recombinant and/or synthetic nucleic acid encoding the peptide or polypeptide. The peptides and polypeptides of this invention can be obtained by chemical synthesis, by proteolytic cleavage of a polypeptide and/or by synthesis from nucleic acid encoding the peptide or polypeptide.

It is also understood that the peptides and polypeptides of this invention may also contain conservative substitutions where a naturally occurring amino acid is replaced by one having similar properties and which does not alter the function of the polypeptide. Such conservative substitutions are well known in the art. Thus, it is understood that, where desired, modifications and changes, which are distinct from the substitutions which modulate immunogenicity, may be made in the nucleic acid and/or amino acid sequence of the peptides and polypeptides of the present invention and still obtain a peptide or polypeptide having like or otherwise desirable characteristics. Such changes may occur in natural isolates or may be synthetically introduced using site-specific mutagenesis, the procedures for which, such as mis-match polymerase chain reaction (PCR), are well known in the art. One of skill in the art will also understand that polypeptides and nucleic acids that contain modified amino acids and nucleotides, respectively (e.g., to increase the half-life and/or the therapeutic efficacy of the molecule), can be used in the methods of the invention.

A recall antigen of this invention is any antigen that activates memory T cells in a subject, as described herein. A recall antigen of this invention can be a whole protein, a fragment of a protein, an immunogenic peptide, an antibody and/or T cell epitope and/or a T cell stimulatory peptide. Identification of immunogenic peptides, T cell stimulatory peptides, antibody and T cell epitopes and the like is carried out by methods well known in the art.

For example, a recall antigen of this invention can include, but is not limited to, influenza antigens, polio antigens, tetanus toxin and other tetanus antigens, herpes antigens [e.g., CMV, EBV, HSV, VZV (chicken pox virus)], mumps antigens, measles antigens, rubella antigens, diphtheria toxin or other diphtheria antigens, pertussis antigens, hepatitis (e.g., hepatitis A and hepatitis B) antigens, smallpox antigens, adenovirus antigens or any other vaccine antigen or other wide-spread antigen to which a substantial portion of the population has been exposed, as would be recognized in the art. A recall antigen of this invention can also be an antigen identified to activate memory T cells in the subject who is the recipient of the recall antigen and thus can be a “custom antigen” specific for that subject.

In one embodiment of the present invention, the recall antigen comprises, consists essentially of, and/or consists of, the tetanus toxoid factor C (TTFC) antigen. In a further embodiment, the recall antigen comprises, consists essentially of, and/or consists of, the TTFC antigen that comprises, consists essentially of, and/or consists of, a polypeptide comprising, consisting essentially of, and/or consisting of, the amino acid sequence of SEQ ID NO:3. An additional embodiment provides a nucleic acid encoding the TTFC recall antigen, which can be an antigen having the amino acid sequence comprising, consisting essentially of, and/or consisting of, the amino acid sequence of SEQ ID NO:3.

A new antigen of this invention is an antigen to which a subject has little or no prior immune response as described herein. Exemplary new antigens of this invention can include, but are not limited to, cancer antigens, infectious agent antigens, allergic reaction antigens (allergens), transplantation antigens, autoantigens and the like as are known in the art. A new antigen of this invention can be a whole protein, a fragment of a protein, an immunogenic peptide, an antibody and/or T cell epitope and/or a T cell stimulatory peptide. Identification of immunogenic peptides, T cell stimulatory peptides, antibody and T cell epitopes, and the like, is carried out by methods well known in the art.

A cancer antigen (i.e., an antigen specifically associated with cancer cells) of this invention can include, for example, HER2/neu and BRCA1 antigens for breast cancer, MART-1/MelanA, gp100, tyrosinase, TRP-1, TRP-2, NY-ESO-1, CDK-4, β-catenin, MUM-1, Caspase-8, KIAA0205, HPVE7, SART-1, PRAME, and p15 antigens, members of the MAGE family, the BAGE family (such as BAGE-1), the DAGE/PRAME family (such as DAGE-1), the GAGE family, the RAGE family (such as RAGE-1), the SMAGE family, NAG, TAG-72, CA125, mutated proto-oncogenes such as p21ras, mutated tumor suppressor genes such as p53, tumor associated viral antigens (e.g., HPV16 E7), the SSX family, HOM-MEL-55, NY-COL-2, HOM-HD-397, HOM-RCC-1.14, HOM-HD-21, HOM-NSCLC-11, HOM-MEL-2.4, HOM-TES-11, RCC-3.1.3, NY-ESO-1, and the SCP family. Members of the MAGE family include, but are not limited to, MAGE-1, MAGE-2, MAGE-3, MAGE-4 and MAGE-11. Members of the GAGE family include, but are not limited to, GAGE-1, GAGE-6. See, e.g., review by Van den Eynde and van der Bruggen (1997) in Curr. Opin. Immunol. 9: 684-693, Sahin et al. (1997) in Curr. Opin. Immunol. 9: 709-716, and Shawler et al. (1997), the entire contents of which are incorporated by reference herein for their teachings of cancer antigens.

The cancer antigen can also be, but is not limited to, human epithelial cell mucin (Muc-1; a 20 amino acid core repeat for Muc-1 glycoprotein, present on breast cancer cells and pancreatic cancer cells), MUC-2, MUC-3, MUC-18, the Ha-ras oncogene product, carcino-embryonic antigen (CEA), the raf oncogene product, CA-125, GD2, GD3, GM2, TF, sTn, gp75, EBV-LMP 1 & 2, HPV-F4, 6, 7, prostatic serum antigen (PSA), prostate-specific membrane antigen (PSMA), alpha-fetoprotein (AFP), CO17-1A, GA733, gp72, p53, the ras oncogene product, β-HCG, gp43, HSP-70, p17 mel, HSP-70, gp43, HMW, HOJ-1, melanoma gangliosides, TAG-72, mutated proto-oncogenes such as p21ras, mutated tumor suppressor genes such as p53, estrogen receptor, milk fat globulin, telomerases, nuclear matrix proteins, prostatic acid phosphatase, protein MZ2-E, polymorphic epithelial mucin (PEM), folate-binding-protein LK26, truncated epidermal growth factor receptor (EGFR), Thomsen-Friedenreich (T) antigen, GM-2 and GD-2 gangliosides, polymorphic epithelial mucin, folate-binding protein LK26, human chorionic gonadotropin (HCG), pancreatic oncofetal antigen, cancer antigens 15-3,19-9, 549, 195, squamous cell carcinoma antigen (SCCA), ovarian cancer antigen (OCA), pancreas cancer associated antigen (PaA), mutant K-ras proteins, mutant p53, and chimeric protein p210_{BCR-ABL} and tumor associated viral antigens (e.g., HPV16 E7).

The cancer antigen of this invention can also be an antibody produced by a B cell tumor (e.g., B cell lymphoma; B cell leukemia; myeloma; hairy cell leukemia), a fragment of such an antibody, which contains an epitope of the idiotype of the antibody, a malignant B cell antigen receptor, a malignant B cell immunoglobulin idiotype, a variable region of an immunoglobulin, a hypervariable region or complementarity determining region (CDR) of a variable region of an immunoglobulin, a malignant T cell receptor (TCR), a variable region of a TCR and/or a hypervariable region of a TCR. In one embodiment, the cancer antigen of this invention can be a single chain antibody (scFv), comprising linked V_{H}, and V_{L} domains, which retains the conformation and specific binding activity of the native idiotype of the antibody.

The present invention is in no way limited to the cancer antigens listed herein. Other cancer antigens be identified, isolated and cloned by methods known in the art such as those disclosed in U.S. Pat. No. 4,514,506, the entire contents of which are incorporated by reference herein.

The cancer to be treated by administration to a subject of a chimeric polypeptide or nucleotide sequence encoding a chimeric polypeptide of this invention can be, but is not limited to, B cell lymphoma, T cell lymphoma, myeloma, leukemia, hematopoietic neoplasias, thymoma, lymphoma, sarcoma, lung cancer, liver cancer, non-Hodgkins lymphoma, Hodgkins lymphoma, uterine cancer, adenocarcinoma, breast cancer, pancreatic cancer, colon cancer, lung cancer, renal cancer, bladder cancer, liver cancer, prostate cancer, ovarian cancer, primary or metastatic melanoma, squamous cell carcinoma, basal cell carcinoma, brain cancer, angiosarcoma, hemangiosarcoma, head and neck carcinoma, thyroid carcinoma, soft tissue sarcoma, bone sarcoma, testicular cancer, uterine cancer, cervical cancer, gastrointestinal cancer, and any other cancer now known or later identified (see, e.g., Rosenberg (1996) Ann. Rev. Med. 47:481-491, the entire contents of which are incorporated by reference herein).

Infectious agent antigens of this invention can include, but are not limited to, antigenic peptides or proteins encoded by the genomes of Hepadnaviridae including hepatitis A, B, C, D, E, F, G, etc. (e.g., HBsAg, HBcAg, HBeAg); Flaviviridae including human hepatitis C virus (HCV), yellow fever virus and dengue viruses; Retroviridae including human immunodeficiency viruses (HIV) (e.g., gp120, gp160, gp41, an active (i.e., antigenic) fragment of gp120, an active (i.e., antigenic) fragment of gp160 and/or an active (i.e., antigenic) fragment of gp41) and human T lymphotropic viruses (HTLV1 and HTLV2); Herpesviridae including herpes simplex viruses (HSV-1 and HSV-2), Epstein Barr virus (EBV), cytomegalovirus, varicella-zoster virus (VZV), human herpes virus 6 (HHV-6) human herpes virus 8 (HHV-8), and herpes B virus; Papovaviridae including human papilloma viruses; Rhabdoviridae including rabies virus; Paramyxoviridae including respiratory syncytial virus; Reoviridae including rotaviruses; Bunyaviridae including hantaviruses; Filoviridae including Ebola virus; Adenoviridae; Parvoviridae including parvovirus B-19; Arenaviridae including Lassa virus; Orthomyxoviridae including influenza viruses (e.g., NP, HA antigen); Poxyiridae including Orf virus, molluscum contageosum virus, smallpox virus and Monkey pox virus; Togaviridae including Venezuelan equine encephalitis virus; Coronaviridae including corona viruses such as the severe acute respiratory syndrome (SARS) virus; and Picornaviridae including polioviruses; rhinoviruses; orbiviruses; picodnaviruses; encephalomyocarditis virus (EMV); Parainfluenza viruses, adenoviruses, Coxsackieviruses, Echoviruses, Rubeola virus, Rubella virus, human papillomaviruses, Canine distemper virus, Canine contagious hepatitis virus, Feline calicivirus, Feline rhinotracheitis virus, TGE virus (swine), Foot and mouth disease virus, simian virus 5, human parainfluenza virus type 2, human metapneuomovirus, enteroviruses, and any other pathogenic virus now known or later identified (see, e.g., Fundamental Virology, Fields et al., Eds., 3^{rd} ed., Lippincott-Raven, New York, 1996, the entire contents of which are incorporated by reference herein for the teachings of pathogenic viruses).

The antigen of this invention can be an antigenic peptide or protein of a pathogenic microorganism, which can include, but is not limited to, Rickettsia, Chlamydia, Mycobacteria, Clostridia, Corynebacteria, Mycoplasma, Ureaplasma, Legionella, Shigella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., Helicobacter spp., and any other pathogenic microorganism now known or later identified (see, e.g., Microbiology, Davis et al, Eds., 4^{th} ed., Lippincott, New York, 1990, the entire contents of which are incorporated herein by reference for the teachings of pathogenic microorganisms).

Specific examples of microorganisms from which the antigen of this invention can be obtained include, but are not limited to, Helicobacter pylori, Chlamydia pneumoniae, Chlamydia trachomatis, Ureaplasma urealyticum, Mycoplasma pneumoniae, Staphylococcus aureus, Streptococcus pyogenes, Streptococcus pneumoniae, Streptococcus viridans, Enterococcus faecalis, Neisseria meningitidis, Neisseria gonorrhoeae, Treponema pallidum, Bacillus anthracis, Salmonella typhi, Vibrio cholera, Pasteurella pestis (Yersinia pestis), Pseudomonas aeruginosa, Campylobacter jejuni, Clostridium difficile, Clostridium botulinum, Mycobacterium tuberculosis, Borrelia burgdorferi, Haemophilus ducreyi, Corynebacterium diphtheria, Bordetella pertussis, Bordetella parapertussis, Bordetella bronchiseptica, Haemophilus influenza, and enterotoxic Escherichia coli.

In one embodiment of the present invention, the new antigen is the Yersinia pestis antigen, LcrV (V antigen). In a further embodiment, the new antigen is the LcrV antigen that comprises, consists essentially of, and/or consists of, the polypeptide comprising, consisting essentially of, and/or consisting of, the amino acid sequence of SEQ ID NO:4. An additional embodiment of the invention provides a nucleic acid encoding the Y. pestis LcrV antigen, which can be the antigen having the amino acid sequence of SEQ ID NO:4.

Antigens of this invention can be antigenic peptides or proteins from pathogenic protozoa, including, but not limited to, Plasmodium species (e.g., malaria antigens), Babeosis species, Schistosoma species, Trypanosoma species, Pneumocystis carnii, Toxoplasma species, Leishmania species, and any other protozoan pathogen now known or later identified.

Antigens of this invention can also be antigenic peptides or proteins from pathogenic yeast and fungi, including, but not limited to, Aspergillus species, Candida species, Cryptococcus species, Histoplasma species, Coccidioides species, and any other pathogenic fungus now known or later identified.

Specific examples of various antigens of this invention include, but are not limited to, the influenza virus nucleoprotein (residues 218-226; Fu et al. (1997) J. Virol. 71: 2715-2721), antigens from Sendai virus and lymphocytic choriomeningitis virus (An et al. (1997) J. Virol. 71: 2292-2302), the B1 protein of hepatitis C virus (Bruna-Romero et al. (1997) Hepatology 25: 470-477), gp 160 of HIV (Achour et al. (1996) J. Virol. 70: 6741-6750), amino acids 252-260 of the circumsporozoite protein of Plasmodium berghei (Allsopp et al. (1996) Eur. J. Immunol. 26: 1951-1958), the influenza A virus nucleoprotein (residues 366-374; Nomura et al. (1996) J. Immunol. Methods 193: 4149), the listeriolysin 0 protein of Listeria monocytogenes (residues 91-99; An et al. (1996) Infect. Immun. 64: 1685-1693), the E6 protein (residues 131-140; Gao et al. (1995) J. Immunol. 155: 5519-5526) and E7 protein (residues 21-28 and 48-55; Bauer et al. (1995) Scand. J. Immunol. 42: 317-323) of human papillomavirus type 16, the M2 protein of respiratory syncytial virus (residues 82-90 and 81-95; Hsu et al. (1995) Immunology 85: 347-350), the herpes simplex virus type 1 ribonucleotide reductase (Salvucci et al. (1995) J. Gen. Virol. 69: 1122-1131), the rotavirus VP7 protein (Franco et al. (1993) J. Gen. Virol. 74: 2579-2586), P. falciparum antigens (causing malaria) and hepatitis B surface antigen (Gilbert et al. (1997) Nature Biotech. 15: 1280-1283).

Transplantation antigens for use an antigen of this invention include, but are not limited to, different antigenic specificities of HLA-A, B and C Class I proteins. Different antigenic specificities of HLA-DR, HLA-DQ, HLA-DP and HLA-DW Class II proteins can also be used (WHO Nomenclature Committee, Immunogenetics 16:135 (1992); Hensen et al., in Fundamental Immunology, Paul, Ed., pp. 577-628, Raven Press, New York, 1993; NIH Genbank and EMBL data bases).

The present invention also contemplates the use of allergic antigens or allergens, which can include, but are not limited to, environmental allergens such as dust mite allergens; plant allergens such as pollen, including ragweed pollen; insect allergens such as bee and ant venom; and animal allergens such as cat dander, dog dander and animal saliva allergens.

The present invention also provides autoantigens as an antigen of this invention, for example, to enhance self-tolerance to an autoantigen in a subject, such as an elderly person, in whom self-tolerance is impaired. Exemplary autoantigens of this invention can include, but are not limited to, myelin basic protein, islet cell antigens, insulin, collagen and human collagen glycoprotein 39, muscle acetylcholine receptor and its separate polypeptide chains and peptide epitopes, glutamic acid decarboxylase and muscle-specific receptor tyrosine kinase.

The present invention also provides a composition comprising a nucleic acid encoding a chimeric polypeptide of this invention. The chimeric polypeptide of this invention can comprise any recall antigen and any new antigen as described herein. It is also to be understood that the recall antigen of this invention can be any antigen described herein or otherwise identified to meet the criteria of a recall antigen as defined herein. Furthermore, a new antigen of this invention can be any antigen described herein or otherwise identified to meet the criteria of a new antigen as defined herein.

One embodiment of the present invention provides a chimeric polypeptide, comprising, consisting essentially of, and/or consisting of, a recall antigen and a new antigen, wherein the recall antigen comprises, consists essentially of, and/or consists of, the TTFC antigen and the new antigen comprises, consists essentially of, and/or consists of, the LcrV antigen. A further embodiment provides a chimeric polypeptide comprising, consisting essentially of, and/or consisting of, a recall antigen and a new antigen, wherein the recall antigen comprises, consists essentially of, and/or consists of, the polypeptide comprising, consisting essentially of, and/or consisting of, the amino acid sequence of SEQ ID NO:3 and the new antigen comprises, consists essentially of, and/or consists of, the polypeptide comprising, consisting essentially of, and/or consisting of, the amino acid sequence of SEQ ID NO:4. A still further embodiment provides a chimeric polypeptide comprising, consisting essentially of, and/or consisting of, a recall antigen and a new antigen, wherein the recall antigen comprises, consists essentially of, and/or consists of, the polypeptide comprising, consisting essentially of, and/or consisting of, the amino acid sequence of SEQ ID NO:3 and the new antigen comprises, consists essentially of, and/or consists of, the polypeptide comprising, consisting essentially of, and/or consisting of, the amino acid sequence of SEQ ID NO:4, and further wherein, the recall antigen and the new antigen are linked by the amino acid sequence of SEQ ID NO:5 or SEQ ID NO:6. Another embodiment of the present invention provides a chimeric polypeptide comprising, consisting essentially of, and/or consisting of, a recall antigen and a new antigen, wherein the chimeric polypeptide comprises, consists essentially of, and/or consists of, the amino acid sequence of SEQ ID NO:7 or SEQ ID NO:9.

Another embodiment of the present invention provides a nucleic acid encoding a chimeric polypeptide comprising, consisting essentially of, and/or consisting of, a recall antigen that reactivates memory T cells in a subject, and a new antigen that activates naïve B cells in a subject. An additional embodiment provides a nucleic acid comprising, consisting essentially of, and/or consisting of, a nucleotide sequence encoding TTFC, which can be the amino acid sequence of SEQ ID NO:3, and/or encoding LcrV, which can be the amino acid sequence of SEQ ID NO:4. The nucleic acid sequence can further comprise, consist essentially of, or consist of, the nucleotide sequence encoding the amino acid sequence of SEQ ID NO:5, SEQ ID NO:6 and/or SEQ ID NO:8. Further provided is a nucleic acid encoding the amino acid sequence of SEQ ID NO:7 and/or SEQ ID NO:9

“Nucleic acid” as used herein refers to single- or double-stranded molecules which may be DNA, comprised of the nucleotide bases A, T, C and G, or RNA, comprised of the bases A, U (substitutes for T), C, and G. The nucleic acid may represent a coding strand or its complement. Nucleic acids may be identical in sequence to the sequence, which is naturally occurring or may include alternative codons, which encode the same amino acid as that which is found in the naturally occurring sequence. Furthermore, nucleic acids may include codons, which represent conservative substitutions of amino acids as are well known in the art. The nucleic acids of this invention can also comprise any nucleotide analogs and/or derivatives as are well known in the art.

As used herein, the term “isolated nucleic acid” means a nucleic acid separated or substantially free from at least some of the other components of the naturally occurring organism, for example, the cell structural components commonly found associated with nucleic acids in a cellular environment and/or other nucleic acids. The isolation of nucleic acids can therefore be accomplished by well-known techniques such as cell lysis followed by phenol plus chloroform extraction, followed by ethanol precipitation of the nucleic acids. The nucleic acids of this invention can be isolated from cells according to methods well known in the art for isolating nucleic acids. Alternatively, the nucleic acids of the present invention can be synthesized according to standard protocols well described in the literature for synthesizing nucleic acids. Modifications to the nucleic acids of the invention are also contemplated, provided that the essential structure and function of the peptide or polypeptide encoded by the nucleic acid are maintained.

The nucleic acid encoding the peptide or polypeptide of this invention can be part of a recombinant nucleic acid construct comprising any combination of restriction sites and/or functional elements as are well known in the art that facilitate molecular cloning and other recombinant DNA manipulations. Thus, the present invention further provides a recombinant nucleic acid construct comprising a nucleic acid encoding a peptide and/or polypeptide of this invention.

The present invention further provides a vector comprising a nucleic acid encoding a peptide and/or polypeptide of this invention. The vector can be any expression vector (e.g., prokaryotic or eukaryotic) which contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise, for example, viral nucleic acid including, but not limited to, vaccinia virus, adenovirus, retrovirus, alphavirus and/or adeno-associated virus nucleic acid. The nucleic acid or vector of this invention can also be in a liposome or a delivery vehicle, which can be taken up by a cell via receptor-mediated or other type of endocytosis.

In some embodiments, the chimeric polypeptide comprises a tag, which facilitates purification. As referred to herein, a “tag” is any added series of amino acids which are provided in a protein at either the C-terminus, the N-terminus, and/or internally. Suitable tags include but are not limited to tags known to those skilled in the art to be useful in purification such as, but not limited to, His tag, glutathione-s-transferase tag, flag tag, mbp (maltose binding protein) tag, etc. Such tagged polypeptides may also be engineered to comprise a cleavage site, such as a thrombin, enterokinase or factor X cleavage site, for ease of removal of the tag before, during and/or after purification.

In one embodiment of the invention, the chimeric polypeptide comprises, consists essentially of, and/or consists, of the amino acid sequence of SEQ ID NO:7, and further wherein a his tag amino acid sequence of SEQ ID NO: 8 is provided at the N-terminus of the amino acid sequence of SEQ ID NO:7. This particular embodiment is also provided by the polypeptide that comprises, consists essentially of, and/or consists, of the amino acid sequence of SEQ ID NO: 9.

An additional embodiment provides a nucleic acid encoding the amino acid sequence of SEQ ID NO:7, and further wherein a nucleotide sequence encoding a his tag, which can be the amino acid sequence of SEQ ID NO:8 is provided at the 5′ end of the nucleotide sequence. This particular embodiment is also provided by nucleic acid that encodes the amino acid sequence of SEQ ID NO: 9.

In one embodiment of the present invention, the expression vector used for production of the chimeric polypeptides is pQE-30 (Qiagen). An additional embodiment of the present invention provides a pQE-30 expression vector, wherein the expression vector comprises a nucleotide sequence cloned in-frame into the BamH1 cloning site, wherein the nucleotide sequence encodes the amino acid sequence of SEQ ID NOs:3, 4, 5, 6, 7, 8, or 9, or any combination thereof.

The nucleic acid of this invention can be in a cell, which can be a cell expressing the nucleic acid whereby a peptide and/or polypeptide of this invention is produced in the cell. In addition, the vector of this invention can be in a cell, which can be a cell expressing the nucleic acid of the vector whereby a peptide and/or polypeptide of this invention is produced in the cell. It is also contemplated that the nucleic acids and/or vectors of this invention can be present in a host (e.g., a bacterial cell, a cell line, a transgenic animal, etc.) that can express the peptides and/or polypeptides of the present invention.

For production of the chimeric polypeptides and/or peptides of this invention in prokaryotes, there are numerous E. coli (Escherichia coli) expression vectors known to one of ordinary skill in the art useful for the expression of nucleic acid encoding proteins such as fusion or chimeric proteins. Other microbial hosts suitable for use include bacilli, such as Bacillus subtilis, and other enterobacteria, such as Salmonella, Serratia, as well as various Pseudomonas species. These prokaryotic hosts can support expression vectors that will typically contain sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence and have ribosome binding site sequences for example, for initiating and completing transcription and translation. If necessary, an amino terminal methionine can be provided by insertion of a Met codon 5′ and in-frame with the coding sequence of the protein. Also, the carboxy-terminal extension of the protein can be removed using standard oligonucleotide mutagenesis procedures.

Additionally, yeast expression systems and baculovirus systems, which are well known in the art, can be used to produce the chimeric peptides and polypeptides of this invention.

The vectors of this invention can be transferred into a cell by well-known methods, which vary depending on the type of cell host. For example, calcium chloride transfection is commonly utilized for prokaryotic cells, whereas calcium phosphate treatment, lipofection or electroporation can be used for other cell hosts.

The nucleic acid encoding the peptides and polypeptides of this invention can be any nucleic acid that functionally encodes the peptides and polypeptides of this invention. To functionally encode the peptides and polypeptides (i.e., allow the nucleic acids to be expressed), the nucleic acid of this invention can include, for example, antibiotic resistance markers, origins of replication and/or expression control sequences, such as, for example, a promoter (constitutive or inducible), an enhancer and necessary information processing sites, such as ribosome binding sites, RNA splice sites, polyadenylation sites and transcriptional terminator sequences.

Examples of expression control sequences useful in this invention include promoters derived from metallothionine genes, actin genes, immunoglobulin genes, CMV, SV40, adenovirus, bovine papilloma virus, etc. A nucleic acid encoding a selected peptide or polypeptide can readily be determined based upon the genetic code for the amino acid sequence of the selected peptide or polypeptide and many nucleic acids will encode any selected peptide or polypeptide. Modifications in the nucleic acid sequence encoding the peptide or polypeptide are also contemplated. Modifications that can be useful are modifications to the sequences controlling expression of the peptide or polypeptide to make production of the peptide or polypeptide inducible or repressible as controlled by the appropriate inducer or repressor. Such methods are standard in the art. The nucleic acid of this invention can be generated by means standard in the art, such as by recombinant nucleic acid techniques and by synthetic nucleic acid synthesis or in vitro enzymatic synthesis.

In certain embodiments of this invention, a chimeric polypeptide and a nucleic acid encoding a chimeric polypeptide can be combined with or without an adjuvant (which can either a polypeptide or a nucleic acid encoding a polypeptide) in any combination that results in administration to a subject of a recall antigen and a new antigen.

In addition, the present invention provides a composition comprising a chimeric polypeptide of this invention and a composition comprising an adjuvant in the form of an amino acid sequence, as well as a nucleic acid encoding a chimeric polypeptide of this invention and a nucleic acid encoding an adjuvant. The adjuvant, in the form of an amino acid sequence, can be a component of the chimeric polypeptide and/or a separate component of the composition comprising the chimeric polypeptide of this invention. The adjuvant in the form of a nucleic acid, can be a component of the nucleic acid encoding the chimeric polypeptide and/or a separate component of the composition comprising the nucleic acid encoding the chimeric polypeptide of this invention. An adjuvant of this invention can be an amino acid sequence that is a peptide, a protein fragment or a whole protein that functions as the adjuvant, or the adjuvant can be a nucleic acid encoding a peptide, protein fragment or whole protein that functions as an adjuvant. As used herein, “adjuvant” describes a substance, which can be any immunomodulating substance capable of being combined with the polypeptide or nucleic acid vaccine to enhance, improve or otherwise modulate an immune response in a subject without deleterious effect on the subject.

An adjuvant of this invention can be, but is not limited to, for example, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algammulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153 (the entire contents of which are incorporated herein by reference), or a less reactogenic composition where the QS21 is quenched with cholesterol as disclosed in PCT publication number WO 96/33739 (the entire contents of which are incorporated herein by reference). A particularly potent adjuvant formulation involving QS21 3D-MPL & tocopherol in an oil in water emulsion is described in PCT publication number WO 95/17210 (the entire contents of which are incorporated herein by reference). In addition, the nucleic acid of this invention can include an adjuvant by comprising a nucleotide sequence encoding a recall antigen and a new antigen of this invention and a nucleotide sequence that provides an adjuvant function, such as CpG sequences. Such CpG sequences, or motifs, are well known in the art.

An adjuvant of this invention, such as, for example, an immunostimulatory cytokine, can be administered before, concurrent with, and/or within a few hours, several hours, and/or 1, 2, 3, 4, 5, 6, 7, 8, 9, and/or 10 days before or after the administration of a composition of this invention to a subject.

Furthermore, any combination of adjuvants, such as immunostimulatory cytokines, can be co-administered to the subject before, after or concurrent with the administration of a composition of this invention. For example, combinations of immunostimulatory cytokines, can consist of two or more immunostimulatory cytokines of this invention, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

Pharmaceutical compositions comprising a composition of this invention and a pharmaceutically acceptable carrier are also provided. The compositions described herein can be formulated for administration in a pharmaceutical carrier in accordance with known techniques. See, e.g., Remington, The Science And Practice of Pharmacy (latest edition). In the manufacture of a pharmaceutical composition according to embodiments of the present invention, the composition of this invention is typically admixed with, inter alia, a pharmaceutically acceptable carrier. By “pharmaceutically acceptable carrier” is meant a carrier that is compatible with other ingredients in the pharmaceutical composition and that is not harmful or deleterious to the subject. The carrier may be a solid or a liquid, or both, and is preferably formulated with the composition of this invention as a unit-dose formulation, for example, a tablet, which may contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and transdermal administration, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for oral administration can be presented in discrete units, such as capsules, cachets, lozenges, or tables, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which may contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The compositions can be presented in unit\dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

“Effective amount” refers to an amount of a compound or composition of this invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)). As a general proposition, a dosage from about 0.01 μg/kg to about 50 mg/kg will have therapeutic efficacy, with all weights being calculated based upon the weight of the composition.

Also as used herein, the terms “treat,” “treating” or “treatment” refer to any type of action that imparts a modulating effect, which, for example, can be a beneficial and/or therapeutic effect, to a subject afflicted with a condition, disorder, disease or illness, including, for example, improvement in the condition of the subject (e.g., in one or more symptoms), delay in the progression of the disorder, disease or illness, prevention or delay of the onset of the disease, disorder, or illness, and/or change in clinical parameters of the condition, disorder, disease or illness, etc., as would be well known in the art.

The frequency of administration of a composition of this invention can be as frequent as necessary to impart the desired therapeutic effect. For example, the composition can be administered one, two, three, four or more times per day, one, two, three, four or more times a week, one, two, three, four or more times a month, one, two, three or four times a year or as necessary to control the condition. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

The compositions of this invention can be administered to a cell of a subject either in vivo or ex vivo. For administration to a cell of the subject in vivo, as well as for administration to the subject, the compositions of this invention can be administered, for example as noted above, orally, parenterally (e.g., intravenously), by intramuscular injection, intradermally (e.g., by gene gun), by intraperitoneal injection, subcutaneous injection, transdermally, extracorporeally, topically or the like. Also, the composition of this invention may be pulsed onto dendritic cells, which are isolated or grown from patient cells, according to methods well known in the art, or onto bulk PBMC or various cell subfractions thereof from a patient.

If ex vivo methods are employed, cells or tissues can be removed and maintained outside the body according to standard protocols well known in the art while the compositions of this invention are introduced into the cells or tissues. For example, the nucleic acids and vectors of this invention can be introduced into cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject. Thus, in one embodiment of this invention, the chimeric polypeptide comprising the recall antigen and new antigen of this invention can be presented to the immune system in a subject on the surface of a cell (i.e., as a cell surface antigen present in the plasma membrane of the cell) and in other embodiments can be presented to the immune system in a subject as a non-cell associated (i.e., cell-free) chimeric polypeptide.

Administration of the nucleic acids of this invention can be achieved by any one of numerous, well-known approaches, for example, but not limited to, direct transfer of the nucleic acids, in a plasmid or viral vector, or via transfer in cells or in combination with carriers such as cationic liposomes. Such methods are well known in the art and readily adaptable for use in the methods described herein. Furthermore, these methods can be used to target certain diseases and cell populations by using the targeting characteristics of the carrier, which would be well known to the skilled artisan.

Transfer vectors employed in the methods of this invention can be any nucleotide construct used to deliver nucleic acid into cells, e.g., a plasmid or viral vector, such as a retroviral vector which can package a recombinant retroviral genome (see e.g., Pastan et al., Proc. Natl. Acad. Sci. U.S.A. 85:4486 (1988); Miller et al., Mol. Cell. Biol. 6:2895 (1986)). The recombinant retrovirus can then be used to infect and thereby deliver a nucleic acid of the invention to the infected cells. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other vector system now known or later identified. Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used nucleic acid transfer methods. Appropriate means for transfection, including viral vectors, chemical transfectants, or physico-mechanical methods such as electroporation and direct diffusion of DNA, are described by, for example, Wolff et al., Science 247:1465-1468, (1990); and Wolff, Nature 352:815-818, (1991).

The compositions of this invention can be used in various methods to elicit-an immune response and/or to treat or prevent a disease or disorder. For example, the present invention provides a method of eliciting an immune response to a new antigen in a subject in whom the ability to elicit an immune response to a new antigen is impaired, comprising administering to the subject an effective amount of a composition of this invention.

Also provided is a method of treating a disorder in a subject in need of such treatment and in whom the ability to elicit an immune response to a new antigen is impaired, comprising administering to the subject a treatment effective amount of the composition of this invention.

The disease and/or disorder that can be treated by the methods of this invention can include any disease or disorder that can be treated by modulating an immune response to an antigen of this invention. For example, the methods of the present invention can be used to treat cancer, viral infections, bacterial infections, fungal infections, parasitic infections and/or other diseases and disorders that can be treated by eliciting an immune response in a subject of this invention.

It is also contemplated that the compositions of this invention can be used as a vaccine or prophylactic composition and employed in methods of preventing a disease or disorder in a subject in whom the ability to elicit an immune response to a new antigen is impaired, comprising administering to the subject an effective amount of the composition of this invention. The vaccine can be administered to a subject who is identified to be at risk of contracting a particular disease or developing a particular disorder and in whom the ability to elicit an immune response to a new antigen is impaired. Identification of a subject at risk can include, for example, evaluation of such factors as family history, genetic predisposition, age, environmental exposure, occupation, lifestyle and the like, as are well known in the art. In other embodiments, the compositions of this invention can be administered to a general population of subjects in whom the ability to elicit an immune response to a new antigen is impaired but for whom specific risk factors have not necessarily been identified.

In some embodiments, the composition to be administered to a subject of this invention can comprise a recall antigen that is identified as such because it is a commonly administered antigen, such as tetanus toxoid, diphtheria toxin, measles vaccine antigen, or mumps vaccine antigen, etc. However, in other embodiments, the present invention provides for methods of producing a “custom vaccine” tailored to a specific individual according to what is a recall antigen and a new antigen to that individual. Thus, the present invention further provides a method of eliciting an immune response to a new antigen in a subject in whom the ability to elicit an immune response to a new antigen is impaired, comprising: a) identifying a recall antigen that reactivates memory T cells in the subject; b) optionally identifying a new antigen against which the subject has little or no detectable memory immune response; and c) administering to the subject an effective amount of a composition comprising the recall antigen and/or a nucleic acid encoding the recall antigen of step (a) and a new antigen and/or a nucleic acid encoding a new antigen.

Also provided is a method of treating a disorder in a subject in need of such treatment and in whom the ability to elicit an immune response to a new antigen is impaired, comprising: a) identifying a recall antigen that reactivates memory T cells in the subject; b) optionally identifying a new antigen against which the subject has little or no detectable memory immune response and to which an immune response can be produced that is effective in treating the disorder; and c) administering to the subject an effective amount of a composition comprising the recall antigen and/or a nucleic acid encoding the recall antigen of step (a) and a new antigen and/or a nucleic acid encoding a new antigen.

Furthermore, the present invention provides a method of preventing a disease in a subject in whom the ability to elicit an immune response to a new antigen is impaired, comprising: a) identifying a recall antigen that reactivates memory T cells in the subject; b) optionally identifying a new antigen against which the subject has little or no detectable memory immune response and to which an immune response can be produced that is effective in preventing the disease; and c) administering to the subject an effective amount of a composition comprising the recall antigen and/or a nucleic acid encoding the recall antigen of step (a) and a new antigen and/or a nucleic acid encoding a new antigen.

An additional embodiment of the invention provides a method of eliciting an immune response to a new antigen in a subject, comprising: a) administering to the subject a recall antigen; and b) administering to the subject a nucleic acid encoding a chimeric polypeptide comprising the recall antigen and a new antigen.

Further provided is a method of treating a disorder in a subject in need of such treatment and in whom the ability to elicit an immune response to a new antigen is impaired, comprising: a) administering to the subject a recall antigen; and b) administering to the subject a nucleic acid encoding a chimeric polypeptide comprising the recall antigen and a new antigen.

Additionally provided is a method of preventing a disease in a subject in whom the ability to elicit an immune response to a new antigen is impaired, comprising: a) administering to the subject a recall antigen; and b) administering to the subject a nucleic acid encoding a chimeric polypeptide comprising the recall antigen and a new antigen.

In another embodiment, a method is provided for eliciting an immune response to a new antigen in a subject, comprising: a) administering to the subject a nucleic acid encoding a recall antigen; and b) administering to the subject a chimeric polypeptide comprising the recall antigen and a new antigen.

The present invention further provides a method of treating a disorder in a subject in need of such treatment and in whom the ability to elicit an immune response to a new antigen is impaired, comprising: a) administering to the subject a nucleic acid encoding a recall antigen; and b) administering to the subject a chimeric polypeptide comprising the recall antigen and a new antigen.

An additional embodiment of the invention provides a method of preventing a disease in a subject in whom the ability to elicit an immune response to a new antigen is impaired, comprising: a) administering to the subject a nucleic acid encoding a recall antigen; and b) administering to the subject a chimeric polypeptide comprising the recall antigen and a new antigen.

The identification of a recall antigen and/or a new antigen in a subject of this invention is carried out by methods standard in the art for detecting immune responses, as set forth above. Such assays can also be used, along with evaluation of symptoms and various clinical parameters, to determine the efficacy of administration of a composition of this invention to a subject of this invention. Such symptoms and/or clinical parameters for a given disease or disorder to be treated and/or prevented are well known in the art.

The present invention additionally provides kits comprising the chimeric polypeptide and/or nucleic acid compositions of this invention, with or without an adjuvant and/or a nucleic acid encoding an adjuvant, along with appropriate buffers, diluents, vessels and/or devices, etc. for measuring a specific amount and for administering the compositions to a subject of this invention.

### EXAMPLES

### Example 1

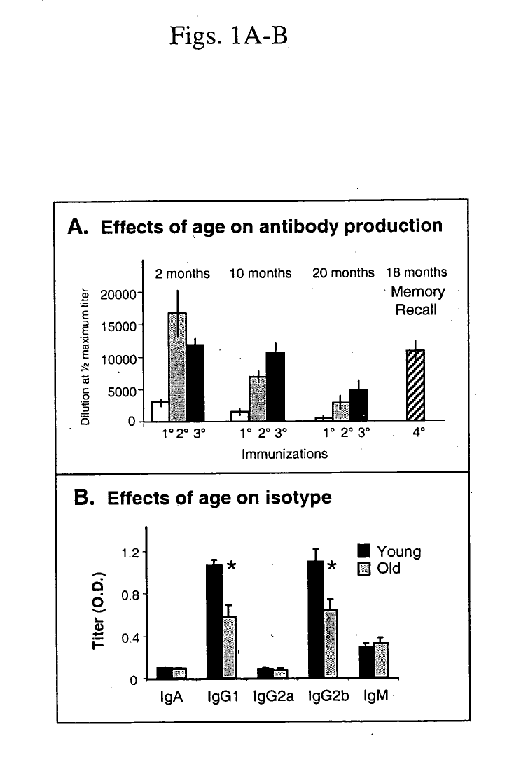

Effects of aging on the B cell response to T-AchR. To evaluate the age-associated decline in susceptibility to experimentally induced myasthenia gravis, the effects of age on the B cell and T cell responses to T-AChR were assessed. Mice in three age groups, 2, 10, and 20 months of age, were immunized with T-AChR in Complete Freund's Adjuvant (CFA). The mice were bled 4 weeks later and sera tested for the presence of antibodies to T-AChR using an ELISA. In response to a primary immunization, there was significantly less anti-T-AChR antibody in older mice (FIG. 1A, left panel). An age-related decline was also seen when responses were measured by specific isotypes, although the overall isotype profile was not altered (FIG. 1B). The decrease in B cell responsiveness to this complex antigen, AChR, was detectable as early as 10-12 months of age. Further studies were then carried out to determine whether there were fewer functional naive B cells in the older mice or that once activated, they were less productive. To determine whether the lower overall responsiveness in older mice could be increased by multiple immunizations that would progressively increase the number of reactive B cells, C57BL/6 mice were boosted monthly. The age-associated decrease in anti-T-AChR titer was still observed after secondary and tertiary immunizations, but was less pronounced (particularly in the middle-aged mice). This indicates that older mice are able to mount a humoral immune response to a complex antigen, like T-AChR, but it is of lower magnitude and may require boosting immunizations in order to reach high levels. These results also suggest that memory B cells are present in older animals and are functional. Moreover, memory T cells, needed to generate the antibody response, must also be present and functional.

The recall memory B cell response. Because B responses were diminished in older mice, experiments were conducted to determine if B cells were stimulated when the mouse was young and then subjected to aging in vivo. For these studies, six young mice (2 months old) were given three immunizations with T-AChR/CFA. They were then allowed to age without additional exposure to T-AChR. After these mice were rested for more than 14 months, sera were taken and tested for residual anti-T-AChR antibody and only a low level was detected. At that time, when the mice were approximately 18 months old, they were given a fourth immunization of AChR/CFA and serum titers were assessed. The memory recall response from the older mice (FIG. 1A, “recall”) was ≈26% lower than the response of the same mice after multiple immunizations at four months of age. By comparison, the average response of 20 month old mice first “seeing” antigen when they were old was 61% lower after tertiary immunization than the two month old controls (FIG. 1A). Thus, the “recall” memory B cells in older mice do not appear to be as affected by the aging process as are naive B cells.

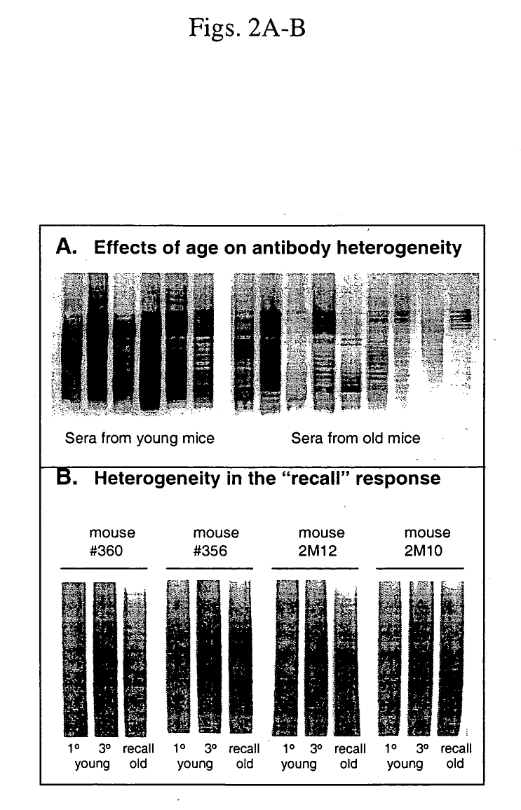

Measurement of anti-T-AChR antibody heterogeneity. The specificities of B cell responses were also addressed by studying the heterogeneity of responding clonotypes. In studies of B cell responsiveness to haptens or small molecules, it appeared that the antibody response becomes more heterogeneous with age (26). To address the question of whether this broadening of the repertoire would also occur in older mice immunized with T-AChR, the diversity of the B cell response was assessed using isoelectric focusing/immunoblotting (47). Briefly, the various antibodies in primary sera from immunized mice were fractionated on a slab isoelectric focusing gel. The antibodies were then electroblotted to nitrocellulose saturated with T-AChR; only those antibodies that bind to T-AChR should stick. The reactive profile was then detected using an enzyme-linked secondary reagent. As shown in FIG. 2A, the response in most young animals was quite diverse; many different T-AChR reactive antibody bands were seen. However, in some, but not all, of the older mice, the reactive antibodies were much more homogeneous. The age-associated narrowing of the B cell repertoire to a complex antigen could affect the ability of the affected animal to adequately develop protective immunity. This analysis was then extended to ask whether heterogeneity was affected by multiple immunizations. When the “recall” sera were tested, they showed a more heterogeneous (youthful) profile, even though there were derived from older animals (FIG. 2B). It appears that T cell memory first induced in response to the earlier immunizations had been key in promoting this effective B cell response.

Summary. Naïve B cell responses to T-AChR were reduced in mice immunized for the first time at an old age. However, when mice were immunized at a young age with T-AChR and allowed to age, the memory B cell responses that were established survived the aging process and could be re-activated to produce a vigorous response in old mice. Since T cell help was critical to the B cell response, memory anti-T-AChR T cells must have also survived aging and provided robust T cell help. This recall response is employed in the present invention to enhance vaccinations in the elderly.

### Example 2

### Studies to Establish that Recall Memory T Cells can Modulate B Cell Responses to Novel Protein Antigens

Rationale. Exposure to antigens not previously encountered often results in reduced naïve and memory immune responses in the elderly. However, studies have shown that when immune memory is established in one's youth, it can survive aging and produce a vigorous recall response in old age (5-9,38). This more “youthful” T cell recall response is harnessed in the present invention to modulate antibody responses in older animals. Old mice will be immunized with a chimeric antigen containing both a carrier that was previously “seen” by the immune system and a “new” antigen against which a modulated antibody response is desired.

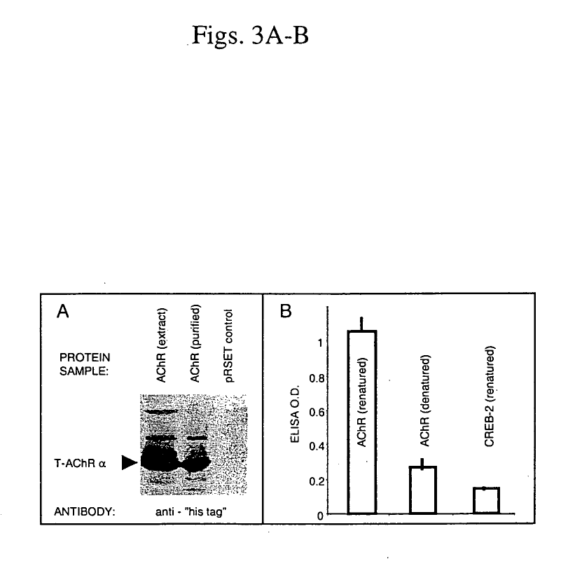

Choice of antigen. T-AChRα will be used as the recall “carrier” antigen. T-AChRα, the extracellular fragment of the α-chain, is routinely produced and renatured according to standard protocols. It has been shown to bind anti-T-AChR antibodies in an ELISA and to produce a robust antibody when injected into C57BL/6 mice (FIG. 3B). It should be noted that although the immunodominant T-AChRα chain has an 80% homology with the mouse AChR, only a very small percentage of the anti-T-AChR antibodies cross react with mouse AChR (48) and the dominant T cell response is not at all cross-reactive (49). Thus, the T-AChR acts like a “foreign” antigen.

Establishing memory responses for later recall. To facilitate the proposed experiments, two month old C57BL/6 mice will be immunized, and then will be aged in order to allow them be sufficiently old (18 months of age). The mice will be immunized in multiple sites (above each shoulder, above each hip, and at the base of the tail) with a total of 10 μg of recombinant T-AChRα fragment or with phosphate buffered saline (PBS) mixed with CFA. The immunizations will be repeated twice at monthly intervals using incomplete Freund's adjuvant (IFA) as adjuvant. The number of mice used was based on an ANOVA power analysis as described herein.

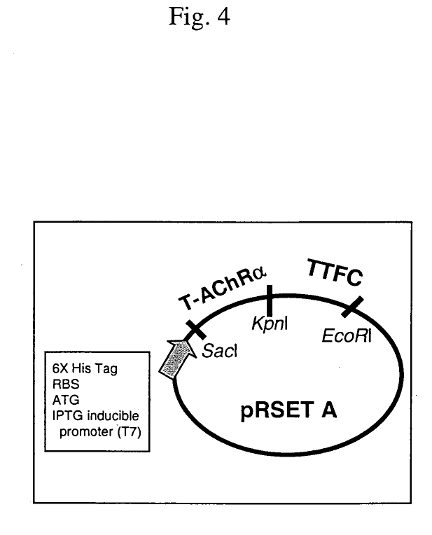

Cloning and expression of test antigens. During the time period that the immunized mice are aging, recombinant DNA technology will be used to generate the antigens for the recall immunizations. The recombinant T-AChRα protein fragment was previously produced by ligating the PCR-generated DNA into pRSET, a prokaryotic expression vector. The same vector will be used to generate all proteins needed for this study. The tetanus toxin fragment C (TTFC) will be generated by PCR using a template of Clostridium tetani DNA and primers sequences specific for the TTFC (described in FIG. 4) (50). The amplified PCR product will be ligated into the prokaryotic expression vector. For production of T-ACRα-TTFC, the TTFC PCR amplicon will be ligated into the previously constructed T-ACRα-containing plasmid, in the proper reading frame so that the expression will produce a chimeric fusion protein. A schematic map of the T-AChRα-TTFC plasmid is shown in FIG. 4. After sequencing to verify proper insertion, the clones will be transformed into E. coli strain BL21 (pLysS). Expression will be induced with IPTG, cells harvested, lysed by sonication, and the fusion protein purified and renatured as done routinely in the preparation of the T-AChRα chain polypeptide. The pRSET vector is designed such that a vector encoded “histidine tag” is included at the amino terminus of the expressed protein to facilitate purification by chromatography on a nickel column.

Testing recall immunity. To test the ability of youthful recall T cell memory responses to provide help to naïve B cells in old animals, the mice will be re-immunized when they reach 17-18 months of age. Those that received T-AChRα at two months of age will be divided into two subgroups; one will be immunized with TTFC and the other with T-AChRα-TTFC. The dosage of antigen in each of the groups will be in equimolar amounts to the 10 μg of T-AChRα received by the mice at earlier immunizations. The same two treatments will be given to the negative control mice that received PBS in their youth. Thus, in total there will be four groups of mice (20 mice per group) in the recall study. The number of mice per group were determined by ANOVA power analysis (power>0.8; p=0.05; minimum detectable difference=0.22; standard deviation=0.2)(51). Previous antibody data from T-AChR immunized mice were used to estimate the standard deviation.

Measuring the B cell response. To determine the effectiveness of recall help in boosting the B cell response in old mice to the newly encountered TTFC antigen, mice will be bled weekly after each immunization and the serum antibody will be tested for titer to both TTFC and T-AChR, for isotype, and for heterogeneity. Sera will be extracted from retro-orbital or tail bleeds and stored at −20° C. until use.

Antibody titers. The titers of antibodies against both antigens will be measured by standard ELISAs as previously described and shown in FIGS. 1 and 3 (52). As an example, in a typical assay, 96 well ELISA plates will be coated with 1 μg per well of either TTFC (the “test” antigen) or T-AChR (the positive control antigen) in 0.05M carbonate/bicarbonate buffer, pH 9.6. After blocking, each test mouse serum, diluted in 1% BSA/PBS, will be added; 5 fold dilutions, from 1:25 to 1:78125, will be used (although any suitable dilution can be used). The secondary antibody will be rabbit anti-mouse IgG (whole molecule) conjugated to horseradish peroxidase (HRP) (Sigma). After development of color, the absorbance at 410 nm will be read on a Dynatech MRX ELISA plate reader. Titers will be plotted graphically, and the dilution at ½ maximum titer will be determined. Sera with known reactivity to tetanus toxoid or to T-ACHR will be used as positive controls. Sera from mice immunized with PBS/CFA will serve as negative controls. It is expected that in old mice, previously immunized with T-AChRα when young, the chimeric T-AChRα-TTFC recall immunization would produce higher anti-TTFC antibody titers than would TTFC alone. In mice immunized with PBS when young, no difference would be expected from immunization in old age with T-AChRα-TTFC in contrast to TTFC alone (since there would have been no memory T cells induced in the young mice immunized with PBS/CFA alone).

Clonotypic patterns. To compare the clonotypic patterns in the anti-TTFC antibody responses between old mice given the recall boost, and old mice given TTFC only, an isoelectric focusing (IEF) immunoblotting procedure will be used (as shown in FIG. 2). Based on ELISA data, approximately equivalent amounts of anti-TTFC Ig will be added to each lane. Following electrophoresis, the focused proteins will be blotted onto a nitrocellulose membrane coated with TTFC. To detect the bound antibodies, the blots are incubated with secondary HRP-conjugated antibody and then developed using diaminobenzidine with H₂O₂. Mice receiving a recall boost from T-AChRα:TTFC immunization would be expected to produce a more heterogeneic pattern of antibody response to TTFC than mice receiving TTFC alone.

Assessing the effects of recall memory on the T cell cytokines. In analyzing the data from this study, the ability of cytokine help produced from the recall response (anti-T-AChRα) to provide a boost to the B cell response to newly encountered antigen (anti-TTFC) will be assessed. Thus, cytokine profiles from T cells of old mice immunized with T-AChRα-TTFA or TTFA alone will be examined and these profiles will be correlated with their respective antibody responses. Assessing cytokine profiles is complicated by the desirability of isolating the response to antigen-specific responsive T cells in a milieu of lymph node cells. T cells will be isolated by a negative selection technique (Stem Cell Technologies) and stimulate in vitro with appropriate antigens to expand the specifically responding clones. For example, in recall mice immunized with T-AChRα-TTFA, cytokine production of the population directed against TTFC will be assessed and compared with the population directed against T-AChRα. Toward this end, after serum samples are taken at 10 days post recall, mice from each of the subgroups having received T-AChRα when young, will be euthanized and the draining lymph nodes removed and teased into cell suspensions in appropriate tissue culture media. T cells will be isolated from lymph node cell (LNC) suspensions using negative selection on a magnetic column (StemCell Technologies). In this technique, unwanted cells are removed from the suspension using magnetically tagged monoclonal antibodies and the CD3+ T cell content of cells recovered from the column is usually 94-98%. The isolated T cells will be mixed with irradiated spleen cells (antigen presenting cells) from syngeneic unimmunized mice and plated in triplicate wells at 200 μl per well. The cells will be stimulated with varying concentrations of the antigens: T-AChRα-TTFA, TTFA alone, T-AChRα alone, or no antigen and incubated at 37 C in 5% CO₂. After 42-48 hours, 50 μl of supernatant will be removed from each well and triplicate samples will be combined and analyzed. Despite the reduced cell count previously encountered in nodes of old mice, it is anticipated that these tests can be run on an individual animal basis, based upon the number of draining lymph nodes, and the number of immunizations given.

BD PharMingen Mouse Th1/Th2 Cytokine CBA kits will be used to measure the Th/1-type cytokines, IL-2, TNF-α, and IFN-γ, and the Th/2-type cytokines, IL-4 and IL-5. This high throughput system allows the simultaneous analysis of five different cytokines based upon a sample size of only 50 μl. Basically, there are five different bead groups, each with distinct fluorescence, and each group has been coated with specific capture antibodies. All five bead specificities are mixed with each test sample and the PE detection reagent is added. After incubation and washing, the labeled beads are analyzed on a Becton Dickinson FACStar Plus. Generation of a standard curve allows data to be reported in pg/ml of sample. Effective help is expected to be seen in conjunction with the production of Th1-type cytokines. Thus, it is expected that T cells from the recall mice (immunized with T-AChR when young) will make high levels of IFN and IL-2, while T cells from the other old animals will have largely switched to IL-4 production.

### Example 3

### Assessment of Whether the Response in Old Mice to the New Antigen can be Modulated by Delivery Through a DNA Vaccine that Encodes the Same T-AChR-TTFC Chimeric Protein

Rationale. The efficacy of a chimeric DNA vaccine will be tested. The vaccine will contain the coding regions T-AChR, a protein that was previously “seen” by the immune system and TTFC, and a new antigen against which a modulated antibody response is desired. Because identical immunogens will be used, a direct comparison of the protein vaccines described in Example 2 and the DNA vaccines of Example 3 will be made of the ability of both vaccines to generate an effective recall response in the elderly.

Establishing memory responses for later recall. The basic strategy of the experiments set forth in Example 3 will be identical to that already described in Example 2. The memory response will be established by immunizing two month old C57BL/6 mice in multiple sites with a total of 10 μg of recombinant T-AChRα fragment or with phosphate buffered saline (PBS) mixed with CFA. The immunizations will be repeated twice at monthly intervals, using IFA as adjuvant.

Cloning of the chimeric AChR-TTFC DNA expression vector. The T-AChRα:TTFC DNA (FIG. 4) will be excised from pRSETA and ligated into pCDNA3.1 (Invitrogen). pCDNA3.1 is a mammalian expression vector in which the expression of protein is driven by a cytomegalovirus promoter (53). In addition, a second construct containing the TTFC DNA fragment alone in the pCDNA3.1 vector will be produced in a similar manner. The correct insertion of the constructs will be verified by sequencing. The ability of the vector constructs to direct protein production will be verified by transfecting the constructs into a mammalian cell line (3T3) and testing for the presence of the protein using TTFC-specific and T-AChRα-specific antibodies in a Western blot assay. Once protein production is verified, the T-AChR-TTFC DNA expression vector will be injected into the hind leg quadriceps muscles of mice and recall immunity will be tested as described herein. The chloramphenicol acetyltransferase (CAT) protein expression plasmid pCDNA3.1/CAT (Invitrogen) will be injected in control mice to verify vaccination techniques.

Testing recall immunity. To test the ability of youthful recall memory responses to boost responses of naïve B cells in old animals, the mice that were immunized with T-AChRα protein or PBS at two months of age will be immunized i.m. with the DNA constructs when they reach approximately 18 months of age. They will receive 100 μg of DNA of either the T-AChRα:TTFC construct or the construct containing TTFC only. As described in Example 2, each of the four subgroups will contain 20 mice based upon an ANOVA power analysis.

Measuring the B cell response. To determine the effectiveness of the DNA vaccine in activating recall help and boosting the B cell response in old mice to the TTFC antigen, blood samples will be taken weekly after immunizations. Based on reports from other models, the highest titers generated from DNA vaccination are typically found from 4-12 weeks post immunization (40). The sera will be collected from retro-orbital or tail vein bleeds and stored at −20° C. until use.

Antibody titers. The titers of anti-TTFC and anti-T-AChRα antibodies will be measured by ELISA as described herein. Effective recall boosting of B cells directed at TTFC will be reflected in higher titers of anti-TTFC antibodies in the T-AChR-TTFC immunized recall mice compared with the mice given TTFC only. The kinetics and titers of antibody production will be analyzed to determine if they are differentially regulated by immunization with a chimeric DNA construct as compared with a chimeric protein.

Clonotypic patterns. The heterogeneity of the antibodies produced will also be measured as described herein. DNA vaccine will be compared with protein vaccine to determine if there is an alteration in the patterns seen with the respective vaccines. A more heterogeneous antibody response would be more indicative of a youthful type of antibody recall.

Assessing the effects of recall memory on T cell cytokines. A comparison of the cytokines produced by T cells from the older “recall” mice immunized with the DNA vaccine versus the protein chimera will also be done. Intramuscularly delivered DNA vaccines have been reported to promote more Th1 type responses. Cytokine profiles of antigen-specific responsive T cells will be assessed as described herein. Increased production of Th1 type cytokines in the T cells from the DNA immunized mice is expected in comparison with the protein immunized mice. Correlating these differences with the corresponding the B cell responses will assist in a determination of what type of T cell help is most effective.

### Example 4

### Immunization of Baboons with a Composition of this Invention

Thirty-two baboons that had been vaccinated when young (with tetanus, for example) are being tested for the ability to give a “recall” response when old (e.g., about 15 to 20 years of age). The old baboons were immunized, with either: i) a chimeric antigen containing a component(s) of the vaccine administered when they were young linked to a “new” test antigen; or ii) the “new” antigen only. Blood samples are being drawn at intervals (e.g., weekly, monthly, etc.) and tested for serum antibody titers to the “new” antigen. For example, as a test antigen, the LcrV protective antigen from Yersinia pestis is being used as a new antigen and tetanus toxoid fragment C (TTFC) as the recall antigen. A synthetic LcrV gene identical in sequence to the published LcrV sequence of the Kimberly strain of Yersinia pestis was cloned in-frame to the tetanus toxoid fragment C. The sequence encoding the cleavage motif for enterokinase was inserted between the LcrV and TTFC genes, to allow for the enzymatic cleavage of LcrV and TTFC polypeptide, if needed. The nucleotide sequence for the chimeric protein antigen (LcrV fused to TTFC) and the LcrV gene alone were cloned in-frame into the Bam H1 cloning site of the prokaryotic expression vector, pQE-30 (Qiagen). The plasmids were transformed into E. coli XL-1Blue cells and expression was induced. The proteins were purified by nickel chromatography (binds the amino-terminal “his” tag). Baboons were immunized by intramuscular injection of 100 micrograms of the LcrV vaccine absorbed to alum or the (LcrV) molar equivalent of the LcrV-TTFC recall chimeric antigen in alum. Before immunization, the animals were bled and tested by ELISA for titers to TTFC. The baboons will be boosted with an injection of 100 micrograms of LcrV absorbed to alum approximately 20 weeks later. The bleeding schedule for these animals is two weeks, and six weeks, following immunization. ELISAs will be performed to assess the antibody titers to LcrV. In addition, antibody affinities and neutralizing potentials will be assessed. It is expected that old animals that received Tetanus immunization when young and were later immunized with the LcrV-TTFC polypeptide will have an altered anti-LcrV response relative to old animals immunized with LcrV alone.

### Example 5

### Comparison of Immunization of Mice Using a Protein Vaccine or a DNA Vaccine

DNA vaccines can be applied either alone or in combination with a protein vaccine protocol (prime with one and boost with the other). Thus, in order to compare protein and DNA vaccines for the ability to modulate an immune response when given as a boost, 80 mice were immunized with either TTFC/alum or with PBS/alum. These animals are now aging and will be challenged with one of the following four antigen preparations: 1) chimeric protein (TTFC-LcrV), 2) chimeric DNA (TTFC-LcrV in a eukaryotic expression vector), 3) new antigen alone (recombinant LcrV protein), 4) new DNA vaccine alone (LcrV in a eukaryotic expression vector). To test the ability of the chimeric protein or DNA vaccine recall immunization to modify the immune response to LcrV, the new antigen, in those animals previously exposed to tetanus (TTFC is the recall antigen), serum will be collected from mice prior to immunization and at weekly intervals thereafter. The serum will be tested to determine the nature (e.g., titers, protective index, and affinities) of the immune response (e.g. antibodies produced) to LcrV.

### Example 6

### Immunization of a Human Subject with a Chimeric Polypeptide of this Invention

As a particular example, a nucleic acid comprising a chimeric polypeptide of this invention, comprising tetanus toxoid fragment C (TTFC) as the recall antigen and Anthrax protective antigen (PA) as the new antigen can be produced according to standard protocols. The nucleic acid can be expressed to produce the chimeric polypeptide that can be administered to a human subject in a pharmaceutically acceptable carrier (e.g., pyrogen-free saline, pH 7.0), with alum as an adjuvant in a dosage of about 15 to about 50 μg in 0.5 ml of carrier. The polypeptide can be administered to a human subject between the ages of 65-85 as a single dose and a second dose can be administered several weeks later (e.g., 14 weeks after the initial dose). Efficacy of the immunization can be determined by detecting the production of protective antibodies in the subject at biweekly intervals for up to a year after the initial immunization.

Alternatively, the nucleic acid encoding the chimeric polypeptide can be administered to the subject as described above according to standard protocols for administration of nucleic acid vaccines as are well known in the art. Efficacy of the immunization can be determined by detecting the production of protective antibodies in the subject as noted above.

Clinical trials employing the chimeric polypeptides and/or nucleic acids of this invention can be carried according to standard protocols, such as those described for example in Gu et al. (“Phase I study of a lipopolysaccharide-based conjugate vaccine against nontypeable Haemophilus influenzae.” Vaccine 21:2107-2114 (2003)), the entire contents of which are incorporated by reference herein for the teachings of clinical studies.

Although the present process has been described with reference to specific details of certain embodiments thereof, it is not intended that such details should be regarded as limitations upon the scope of the invention except as and to the extent that they are included in the accompanying claims.

Throughout this application, various publications are referenced. The disclosures of these publications in their entireties are hereby incorporated by reference into this application in order to more fully describe the state of the art to which this invention pertains.

### REFERENCES

1. Weksler, M. E. 2000. Changes in the B-cell repertoire with age. Vaccine 18:1624.
2. Weksler, M. E. and P. Szabo. 2000. The effect of age on the B-cell repertoire. J. Clin. Immunol. 20:240.
3. Miller, R. A. 1996. The aging immune system: primer and prospectus. Science 273:70.
4. Miller, R. 1995. Immune System. In Handbook of Physiology: Aging. E. J. Masoro, ed. Oxford Press, NY, p. 555.
5. Looney, R. J., M. S. Hasan, D. Coffin, D. Campbell, A. R. Falsey, J. Kolassa, J. M. Agosti, G. N. Abraham, and T. G. Evans. 2001. Hepatitis B immunization of healthy elderly adults: relationship between naive CD4+ T cells and primary immune response and evaluation of GM-CSF as an adjuvant. J. Clin. Immunol. 21:30.
6. Sanchez, M., K. Lindroth, E. Sverremark, F. A. Gonzalez, and C. Fernandez. 2001. The response in old mice: positive and negative immune memory after priming in early age. Int. Immunol. 13:1213.
7. Harrod, T., M. Martin, and M. W. Russell. Long-term persistence and recall of immune responses in aged mice after mucosal immunization. Oral Microbiol. & Immunol. 2001 Jun.; 16.(3):170.-7. 16:170.
8. Sambhara, S., A. Kurichh, R. Miranda, O. James, B. Underdown, M. Klein, J. Tartaglia, and D. Burt. 2001. Severe impairment of primary but not memory responses to influenza viral antigens in aged mice: costimulation in vivo partially reverses impaired primary immune responses. Cell. Immunol. 210:1.
9. Stacy, S., K. A. Krolick, A. J. Infante, and E. Kraig. 2002. Immunological memory and late onset autoimmunity. Mech. Ageing. Dev. 123:975.
10. Schwab, R., C. A. Walters, and M. E. Weksler. 1989. Host defense mechanisms and aging. Semin. Oncol. 16:20.
11. Makinodan, T. and M. M. Kay. 1980. Age influence on the immune system. Adv. Immunol. 29:287.
12. Bovbjerg, D. H., Y. T. Kim, R. Schwab, K. Schmitt, T. DeBlasio, and M. E. Weksler. 1991. “Cross-wiring” of the immune response in old mice: increased autoantibody response despite reduced antibody response to nominal antigen. Cell. Immunol. 135:519.
13. George, A. J. and M. A. Ritter. 1996. Thymic involution with ageing: obsolescence or good housekeeping? Immunol. Today 17:267.
14. Douek, D. C., R. D. McFarland, P. H. Keiser, E. A. Gage, J. M. Massey, B. F. Haynes, M. A. Polis, A. T. Haase, M. B. Feinberg, J. L. Sullivan, B. D. Jamieson, J. A. Zack, L. J. Picker, and R. A. Koup. 1998. Changes in thymic function with age and during the treatment of HIV infection. Nature 396:690.
15. Linton, P. J., L. Haynes, L. Tsui, X. Zhang, and S. Swain. 1997. From naive to effector—alterations with aging. Immunol. Rev. 160:9.
16. Rink, L., I. Cakman, and H. Kirchner. 1998. Altered cytokine production in the elderly. Mech. Ageing Dev. 102:199.
17. Kubo, M. and B. Cinader. 1990. Polymorphism of age-related changes in interleukin (IL) production: differential changes of T helper subpopulations, synthesizing IL 2, IL 3 and IL 4. Eur. J. Immunol. 96: 1289.
18. Miller, R. A. and O. Stutman. 1981. Decline, in aging mice, of the anti-2,4,6-trinitrophenyl (TNP) cytotoxic T cell response attributable to loss of Lyt-2-, interleukin 2-producing helper cell function. Eur. J. Immunol. 6:751.
19. Miller, R. A., G. Garcia, C. J. Kirk, and J. M. Witkowski. 1997. Early activation defects in T lymphocytes from aged mice. Immunol. Rev. 160:79.
20. Kirschmann, D. A. and D. M. Murasko. 1992. Splenic and inguinal lymph node T cells of aged mice respond differently to polyclonal and antigen-specific stimuli. Cell. Immunol. 139:426.
21. Utsuyama, M., Z. Varga, K. Fukami, Y. Homma, T. Takenawa, and K. Hirokawa. 1993. Influence of age on the signal transduction of T cells in mice. Int. Immunol. 5:1177.
22. Fulop, T. J., D. Gagne, A. C. Goulet, S. Desgeorges, G. Lacombe, M. Arcand, and G. Dupuis. 1999. Age-related impairment of p56lck and ZAP-70 activities in human T lymphocytes activated through the TcR/CD3 complex. Exp. Gerontol. 34:197.
23. Engwerda, C. R., B. S. Handwerger, and B. S. Fox. 1994. Aged T cells are hyporesponsive to costimulation mediated by CD28. J. Immunol. 152:3740.
24. Trebilcock, G. U. and U. Ponnappan. 1998. Nuclear factor-kappaB induction in CD45RO+ and CD45RA+ T cell subsets during aging. Mech. Ageing Dev. 102:149.
25. Eisenbraun, M. D., A. Tamir, and R. A. Miller. 2000. Altered composition of the immuno-logical synapse in an anergic, age-dependent memory T cell subset. J. Immunol. 164:6105.
26. Yang, X., J. Stedra, and J. Cerny. 1996. Relative contribution of T and B cells to hypermutation and selection of the antibody repertoire in germinal centers of aged mice. J. Exp. Med. 183:959.
27. Gahring, L. C. and W. O. Weigle. 1990. The effect of aging on the induction of humoral and cellular immunity and tolerance in two long-lived mouse strains. Cell. Immunol. 128:142.
28. Endoh, M., S. I. Rapoport, and T. Tabira. 1990. Studies of experimental allergic encephalomyelitis in old mice. J. Neuroimmunol. 29:21.
29. Biro, J. and E. Beregi. 1980. The influence of influenza vaccinations on human peripheral blood lymphocytes relating to aging. Aktuelle. Gerontologie. 10:319.
30. Miller, C. and G. Kelsoe. 1995. Ig VH hypermutation is absent in the germinal centers of aged mice. J. Immunol. 155:3377.
31. Nicoletti, C., X. Yang, and J. Cerny. 1993. Repertoire diversity of antibody response to bacterial antigens in aged mice. III. Phosphorylcholine antibody from young and aged mice differs in structure and protective activity against infection with Streptococcus pneumoniae. J. Immunol. 150:543.
32. Stephan, R. P., D. A., and P. L. Witte. 1997. Development of B cells in aged mice: decline in the ability of pro-B cells to respond to IL-7 but not to other growth factors. J. Immunol. 158:1598.
33. Kline, G. H., T. A. Hayden, and N. R. Klinman. 1999. B cell maintenance in aged mice reflects both increased B cell longevity and decreased B cell generation. J. Immunol. 162:3342.
34. Klinman, N. R. and G. H. Kline. 1997. The B-cell biology of aging. Immunol. Rev. 160:103.
35. Fagiolo, U., A. Amadori, R. Biselli, R. Paganelli, R. Nisini, E. Cozzi, R. Zamarchi, and R. D'Amelio. 1993. Quantitative and qualitative analysis of anti-tetanus toxoid antibody response in the elderly. Humoral immune response enhancement by thymostimulin. Vaccine 11:1336.
36. Arreaza, E. E., J. J. Gibbons Jr., G. W. Siskind, and M. E. Weksler. 1993. Lower antibody response to tetanus toxoid associated with higher auto-anti-idiotypic antibody in old compared with young humans. Clin. Exp. Immunol. 92:169.
37. Ahmed, R. and D. Gray. 1996. Immunological memory and protective immunity: understanding their relation. Science 272:54.
38. Bender, B. S., E. Tallman, M. P. Johnson, and P. A. J. Small. 1990. Enhancement of anti-influenza cytotoxic T lymphocyte activity in senescent mice by vaccination early in life. Mech. Ageing Dev. 55:1.
39. Jacobson, R. M. and G. A. Poland. 2002. The pneumococcal conjugate vaccine. Minerva Pediatr. 54:295.
40. Gurunathan, S., D. M. Klinman, and R. A. Seder. 2000. DNA vaccines: immunology, application, and optimization*. Annu. Rev. Immunol. 18:927.
41. Ross-Van Eijndhoven, D. G., H. J. Cools, R. G. Westendorp, A. J. Ten Cate-Hoek, D. L. Knook, and E. J. Remarque. 2001. Randomized controlled trial of seroresponses to double dose and booster influenza vaccination in frail elderly subjects. J. Med. Virol. 63:293.
42. Stepanova, L., A. Naykhin, C. Kolmskog, G. Jonson, I. Barantceva, M. Bichurina, O. Kubar, and A. Linde. 2002. The humoral response to live and inactivated influenza vaccines administered alone and in combination to young adults and elderly. J. Clin. Virol. 24:193.
43. Corrigan, E. M. and R. L. Clancy. 1999. Is there a role for a mucosal influenza vaccine in the elderly? Drugs Aging 15:169.
44. Seo, J. Y., S. Y. Seong, B. Y. Ahn, I. C. Kwon, H. Chung, and S. Y. Jeong. 2002. Cross-protective immunity of mice induced by oral immunization with pneumococcal surface adhesin a encapsulated in microspheres. Infect. Immun. 70:1143.
45. Bondada, S., H. Wu, D. A. Robertson, and R. L. Chelvarajan. 2000. Accessory cell defect in unresponsiveness of neonates and aged to polysaccharide vaccines. Vaccine 19:557.
46. Manning, B. M., E. Y. Enioutina, D. M. Visic, A. D. Knudson, and R. A. Daynes. 2001. CpG DNA functions as an effective adjuvant for the induction of immune responses in aged mice. Exp. Gerontol. 37:107.
47. Thompson, P. A. and K. A. Krolick. 1992. Acetylcholine receptor-reactive antibodies in experimental autoimmune myasthenia gravis differing in disease-causing potential: subsetting of serum antibodies by preparative isoelectric focusing. Clin. Immunol. Immunopathol. 62:199.
48. Berman, P. W. and J. Patrick. 1980. Experimental myasthenia gravis. A murine system. J. Exp. Med. 151:204.
49. Wall, K. A., J. Y. Hu, P. Currier, S. Southwood, A. Sette, and A. J. Infante. 1994. A disease-related epitope of Torpedo acetylcholine receptor. Residues involved in I-Ab binding, self-nonself discrimination, and TCR antagonism. J. Immunol. 152:4526.
50. Medaglini, D., A. Ciabattini, M. R. Spinosa, T. Maggi, H. Marcotte, M. R. Oggioni, and G. Pozzi. 2001. Immunization with recombinant Streptococcus gordonii expressing tetanus toxin fragment C confers protection from lethal challenge in mice. Vaccine 19:1931.
51. Glantz, S. A. 1997. Primer of Biostatistics. McGraw-Hill, New York.
52. Mohan, S., R. J. Barohn, C. E. Jackson, and K. A. Krolick. 1994. Evaluation of myosin-reactive antibodies from a panel of myasthenia gravis patients. Clin. Immunol. Immunopathol. 70:266.
53. Stratford, R., G. Douce, F. Bowe, and G. Dougan. 2001. A vaccination strategy incorporating DNA priming and mucosal boosting using tetanus toxin fragment C (TetC). Vaccine 20:516.

What is claimed is:

1. A composition comprising a chimeric polypeptide comprising a recall antigen that reactivates memory T cells in a subject and a new antigen that activates naïve B cells in a subject.

2. The composition of claim 1, wherein the recall antigen is tetanus toxoid fragment C.

3. The composition of claim 1, wherein the new antigen is Yersinia pestis LcrV antigen.

4. The composition of claim 1, wherein the recall antigen is tetanus toxoid fragment C and the new antigen is Yersinia pestis LcrV antigen.

5. The composition of claim 1, further comprising an adjuvant.

6. The composition of claim 1 wherein the recall antigen and new antigen are present in a ratio of 5:1, 4:1, 3:1, 2:1, 1:1, 1:2, 1:3, 1:4 or 1:5.

7. The composition of claim 1 in a pharmaceutically acceptable carrier.

8. The composition of claim 5 in a pharmaceutically acceptable carrier.

9. A composition comprising a nucleic acid encoding a chimeric polypeptide comprising a recall antigen that reactivates memory T cells in a subject and a new antigen that activates naïve B cells in a subject.

10. The composition of claim 9, wherein the recall antigen is tetanus toxoid fragment C.

11. The composition of claim 9, wherein the new antigen is Yersinia pestis LcrV antigen.

12. The composition of claim 9, wherein the recall antigen is tetanus toxoid fragment C and the new antigen is Yersinia pestis LcrV antigen.

13. The composition of claim 9, further comprising a nucleic acid encoding an adjuvant.

14. The composition of claim 9 wherein the nucleotide sequence of the nucleic acid encoding the recall antigen and the nucleotide sequence of the nucleic acid encoding the new antigen are present in a ratio of 5:1, 4:1, 3:1, 2:1, 1:1, 1:2, 1:3, 1:4, or 1:5.

15. The composition of claim 9 in a pharmaceutically acceptable carrier.

16. The composition of claim 13 in a pharmaceutically acceptable carrier.

17. A method of eliciting an immune response to a new antigen in a subject, comprising a) administering to the subject a recall antigen, and b) administering to the subject a nucleic acid encoding a chimeric polypeptide comprising the recall antigen and a new antigen.

18. A method of treating a disorder in a subject in need of such treatment and in whom the ability to elicit an immune response to a new antigen is impaired, comprising administering to the subject an effective amount of the composition of claim 1.

19. A method of eliciting an immune response to a new antigen in a subject in whom the ability to elicit an immune response to a new antigen is impaired, comprising:
a) identifying a recall antigen that reactivates memory T cells in the subject; and
b) administering to the subject an effective amount of a composition comprising a chimeric polypeptide comprising the recall antigen of step (a) and a new antigen.

20. The method of claim 19, further comprising a step (c) of identifying a new antigen against which the subject has little or no detectable memory immune response and administering to the subject an effective amount of a composition comprising a chimeric polypeptide or a nucleic acid encoding a chimeric polypeptide, wherein the chimeric polypeptide comprises the recall antigen of step (a) and the new antigen identified in step (c).


SEQUENCE LISTING (9 sequences)

SEQ ID NO: 1 - DNA, 29 bp, Artificial
  PCR primer
cccaggtacctcaacaccaattccatttt

SEQ ID NO: 2 - DNA, 28 bp, Artificial
  PCR primer
gtagaattctgtccatccttcatctgta

SEQ ID NO: 3 - protein, 451 aa, Clostridium tetani
KNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 4 - protein, 326 aa, Yersinia pestis
MIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGK

SEQ ID NO: 5 - protein, 5 aa, Artificial
  Enterokinase protease recognition sequence
DDDDR

SEQ ID NO: 6 - protein, 15 aa, Artificial
  Linker region sequence
GSHVISSIASDDDDR

SEQ ID NO: 7 - protein, 792 aa, Artificial
  Chimeric LcrV/TTFC polypeptide
MIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGKGSHVISSIASDDDDRKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 8 - protein, 12 aa, Artificial
  N-terminal His tag sequence
MRGSHHHHHHGS

SEQ ID NO: 9 - protein, 804 aa, Artificial
  Chimeric LcrV/TTFC polypeptide with N-terminal His tag
MRGSHHHHHHGSMIRAYEQNPQHFIEDLEKVRVEQLTGHGSSVLEELVQLVKDKNIDISIKYDPRKDSEVFANRVITDDIELLKKILAYFLPEDAILKGGHYDNQLQNGIKRVKEFLESSPNTQWELRAFMAVMHFSLTADRIDDDILKVIVDSMNHHGDARSKLREELAELTAELKIYSVIQAEINKHLSSSGTINIHDKSINLMDKNLYGYTDEEIFKASAEYKILEKMPQTTIQVDGSEKKIVSIKDFLGSENKRTGALGNLKNSYSYNKDNNELSHFATTCSDKSRPLNDLVSQKTTQLSDITSRFNSAIEALNRFIQKYDSVMQRLLDDTSGKGSHVISSIASDDDDRKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND